(19) United States
(12) Patent Application Publication
(10) Pub. No.: US 2026/0070965 A1
(43) Pub. Date: Mar. 12, 2026

(54) ANTI-GDF15 ANTIBODY FOR NEOADJUVANT THERAPY OF CANCER

(71) Applicant: CatalYm GmbH, Planegg-Martinsried (DE)

(72) Inventors: Eugen Leo, München (DE); Frank Hermann, München (DE)

(21) Appl. No.: 19/222,304

(22) Filed: May 29, 2025

Related U.S. Application Data
(63) Continuation of application No. PCT/EP2023/086107, filed on Dec. 15, 2023.

(30) Foreign Application Priority Data
Dec. 16, 2022 (EP) 22214252.3

(51) Int. Cl. C07K 16/22; A61K 39/00; A61K 45/06; A61P 35/00; C07K 16/28

(52) U.S. Cl. CPC C07K 16/22; A61K 45/06; A61P 35/00; C07K 16/2818; C07K 16/2827; A61K 2039/507; A61K 2039/54; A61K 2039/545; C07K 2317/565

(57) ABSTRACT

The present invention relates to an anti-GDF15 antibody for use in a neoadjuvant therapy of cancer.

### CROSS-REFERENCE TO RELATED APPLICATIONS

This application is a continuation of International Patent Application No. PCT/EP2023/086107, filed Dec. 15, 2023, which claims priority from European Patent Application No. 22214252.3, filed Dec. 16, 2022, the contents of which are each hereby incorporated by reference in their entireties.

### REFERENCE TO AN ELECTRONIC SEQUENCE LISTING

This application contains a Sequence Listing which has been submitted electronically in XML format and is hereby incorporated by reference in its entirety. Said XML copy, created on May 12, 2025, is named 217795_seqlist.xml and is 20,480 bytes in size.

### FIELD OF INVENTION

The present invention relates to an anti-GDF15 antibody for use in a neoadjuvant therapy of cancer.

### BACKGROUND

Research in the field of cancer treatment has provided different therapy strategies such as surgery, chemotherapy, radiation therapy, hormonal therapy, targeted therapy (including immunotherapy such as monoclonal antibody therapy) or synthetic lethality which are applied depending inter alia on the location and grade of the tumor and the stage of the disease. Whilst complete removal of the cancer without damage to the rest of the body for example by surgery would be an ideal treatment outcome, the effectiveness of surgical intervention is often limited due to the propensity of cancers to invade adjacent tissue or to spread to distant sites by microscopic metastasis. Neoadjuvant therapy such as chemotherapy or hormonal therapy are given in addition to the primary or initial therapy to promote the reduction of the size of the tumor so as to facilitate a more effective surgery.

For example, urothelial bladder carcinoma is the fourth most common cancer in the United States (US) with over 60,000 new cases each year. Approximately 25% of patients with urothelial carcinoma will have muscle-invasive disease (T2-4) and either present with or later develop metastases (von der Maase et al, 2005). Initially, if in a patient a bladder mass is detected, a transurethral resection of the bladder tumor (TURBT) is conducted for full primary tumor staging. When presence of muscle invasive bladder cancer (MIBC) is confirmed, radical cystectomy (RC) will be performed. MIBC is generally associated with a high rate of recurrence and poor overall prognosis despite aggressive local and systemic treatment strategies. Unfortunately, still up to 50% of patients will have a recurrence of the cancer within 12 months and all patients remain at increased risk of recurrence for the remainder of their lives. Even after RC, the 5-year mortality rate of patients with MIBC remains at about 50%-70% (Grossman et al, 2003).

Owing to this high recurrence rate, neoadjuvant chemotherapy administered in this setting has been well established and ranked on level 1 evidence in the National Comprehensive Cancer Network guidelines (NCCN, 2021), yet only a relatively small survival improvement is achieved over RC alone (approximating 6% at 5 years).

In light of the aggressiveness of MIBC and the poor prognosis together with the high recurrence rate after RC and death rate, there is a need for improved treatment strategies in the art.

Whilst the present invention exemplifies a clinical study with respect to a specific cancer (i.e. MIBC), it is understood that the results will also allow to adapt and improve the treatment of other types of cancer in a human patient in general.

### DESCRIPTION OF THE INVENTION

The present invention aims to overcome the unmet clinical needs to provide a safe and effective therapy for the treatment of cancer in human patients.

Based on extensive experimental tests, the inventors of the present application have found that the anti-growth differentiation factor 15 (GDF-15) antibody visugromab (CTL-002) generates a tumor-selective immune influx of CD8+ and CD4+ T cells. Moreover, visugromab has demonstrated encouraging antitumoral efficacy in combination with anti-PD-1 treatment in fully anti-PD-1/PD-L1 relapsed/refractory subjects (see e.g. WO 2022/101263).

Due to the negative correlation of GDF-15 expression in urothelial tumors with definitive clinical outcome and reduced response to checkpoint-inhibitor treatment, bladder cancer has been identified as an indication of interest. In this respect, the present invention sets out to provide clinical data using visugromab for the neoadjuvant treatment of subjects with MIBC.

Moreover, in a non-limiting embodiment, the present invention also teaches a mode of action of an anti-GDF-15 antibody as neoadjuvant therapy for the treatment of cancer. According to this embodiment of the present invention, the addition of visugromab to/as neoadjuvant treatment can contribute to downstage the tumor and potentially also lead to a pathologic complete response (pCR) in the primary tumor (pTO) in a greater proportion of subjects than with current immune-or chemotherapies alone.

A further non-limiting advantage of the neoadjuvant therapy according to the invention is that it allows to bring immune cells into the tumor very efficiently, because the blood vessels of the tumor are still intact prior to the surgery. The neoadjuvant therapy according to the invention therefore allows to improve immune responses to the cancer.

Accordingly, a neoadjuvant therapy using an anti-GDF-15 antibody may allow to reduce the risk of relapse and relapse-free survival in human cancer patients in general.

The present invention provides the following preferred embodiments:

Item 1 An anti-GDF-15 antibody for use in a neoadjuvant therapy of cancer in a human patient.

Item 2 The anti-GDF-15 antibody for use according to item 1, wherein the cancer is a solid cancer, and wherein neoadjuvant therapy is for treating or inhibiting the growth of a tumor of the solid cancer.

Item 3 The anti-GDF-15 antibody for use according to item 1 or 2, wherein the anti-GDF-15 antibody and at least one checkpoint inhibitor is to be administered in combination, wherein the checkpoint inhibitor is preferably an anti-PD-1 antibody or an anti-PD-L1 antibody.

Item 4 The anti-GDF-15 antibody for use according to item 2 or 3, wherein the patient is scheduled for surgery of a primary tumor of the solid cancer.

Item 5 The anti-GDF-15 antibody for use according to any one of items 2 to 4, wherein the surgery is a resection surgery.

Item 6 The anti-GDF-15 antibody for use according to any one of items 2 to 5, wherein said resection surgery is a radical cystectomy comprising the removal of the bladder, adjacent organs, and regional lymph nodes.

Item 7 The anti-GDF-15 antibody for use according to any one of items 2 to 6, wherein the duration of said neoadjuvant therapy prior to resection surgery is up to 3, 6 or 12 weeks.

Item 8 The anti-GDF-15 antibody for use according to any one of items 2 to 7, wherein the therapy is intended to downstage locally advanced solid cancer to reduce the extent of surgery or to perform an organ-conserving surgery.

Item 9 The anti-GDF-15 antibody for use according to item 8, wherein the duration of said neoadjuvant therapy is up to 3, 4, 6, 12 or 24 weeks.

Item 10 The anti-GDF-15 antibody for use according to any one of items 2 to 9, wherein the cancer is selected from the group consisting of colorectal cancer, gastric cancer, bladder cancer, melanoma, non-small cell lung cancer, head and neck squamous cell cancer, hepatocellular cancer, prostate adenocarcinoma, pancreatic cancer, uterine cancer, cervical cancer, thyroid cancer, cutaneous squamous cell cancer, mesothelioma, cancer of unknown primary (CUP) and breast carcinoma.

Item 11 The anti-GDF-15 antibody for use according to item 10, wherein the cancer is bladder cancer.

Item 12 The anti-GDF-15 antibody for use according to item 11, wherein the bladder cancer is muscle invasive bladder cancer.

Item 13 The anti-GDF-15 antibody for use according to any one of items 1 to 12, wherein the patient cannot receive or refuses to receive chemotherapy.

Item 14 The anti-GDF-15 antibody for use according to item 13, wherein the chemotherapy is a chemotherapy for use in neoadjuvant therapy, such as a cisplatin-based chemotherapy.

Item 15 The anti-GDF-15 antibody for use according to any one of items 2 to 14, wherein a tissue sample of the tumor has a PD-L1 combined positive score (CPS) of ≥10%.

Item 16 The anti-GDF-15 antibody for use according to any one of items 2 to 14, wherein a tissue sample of the tumor has a PD-L1 CPS of <10%.

Item 17 The anti-GDF-15 antibody for use according to any one of items 1 to 16, wherein the cancer is classified as a T2 stage tumor.

Item 18 The anti-GDF-15 antibody for use according to any one of items 1 to 17, wherein the cancer is classified as T3/T4 stage tumor.

Item 19 The anti-GDF-15 antibody for use according to any one of items 1 to 18, wherein the cancer tissue encompasses non-basal/squamous histology.

Item 20 The anti-GDF-15 antibody for use according to any one of items 1 to 18, wherein the cancer tissue encompasses basal/squamous histology.

Item 21 The anti-GDF-15 antibody for use according to any one of items 1 to 20, wherein the anti-GDF-15 antibody is to be administered to the human patient, preferably

at a dose of between 3 and 20 mg/kg, preferably at a dose of 10 mg/kg, and at a dosage regimen of at least one administration cycle, wherein the cycle is a period of two weeks and wherein said dose is to be administered at least once in each of the at least one cycle,

at a dose of between 3 and 20 mg/kg, preferably at a dose of 10 mg/kg, and at a dosage regimen of at least one administration cycle, wherein the cycle is a period of three weeks and wherein said dose is to be administered at least once in each of the at least one cycle, or

at a dose of between 3 and 20 mg/kg, preferably at a dose of 20 mg/kg, and at a dosage regimen of at least one administration cycle, wherein the cycle is a period of four weeks and wherein said dose is to be administered at least once in each of the at least one cycle.

Item 22 The anti-GDF-15 antibody for use according to item 21, wherein the anti-GDF-15 antibody is to be administered at a dose of 20 mg/kg, and at a dosage regimen of at least one administration cycle, wherein the cycle is a period of four weeks and wherein said dose is to be administered at least once in each of the at least one cycle.

Item 23 The anti-GDF-15 antibody for use according to item 21 or 22, wherein the cycle is a period of four weeks, wherein said dose is to be administered at least once in each of the at least one cycle and wherein the dose is to be administered for at least one, two, three or four cycle(s).

Item 24 The anti-GDF-15 antibody for use according to any one of items 21 to 23, wherein the anti-GDF-15 antibody is to be administered to the human patient at a dose of 20 mg/kg, and at a dosage regimen of three administration cycles, wherein the cycle is a period of four weeks and wherein said dose is to be administered at least once in each cycle.

Item 25 The anti-GDF-15 antibody for use according to any one of items 3 to 24, wherein said checkpoint inhibitor is to be administered in the same dosage regimen as the anti-GDF-15 antibody.

Item 26 The anti-GDF-15 antibody for use according to item 25, wherein said checkpoint inhibitor is to be administered prior to the administration of anti-GDF-15 antibody, preferably within 120 min prior to the administration of anti-GDF-15 antibody, more preferably within 30 minutes prior to the administration of anti-GDF-15 antibody.

Item 27 The anti-GDF-15 antibody for use according to items 25 or 26, wherein said dose of the anti-GDF-15 antibody is to be administered intravenously.

Item 28 The anti-GDF-15 antibody for use according to any one of items 1 to 27, wherein the anti-GDF-15 antibody comprises a heavy chain variable domain comprising a CDR1 region represented by an amino acid sequences shown in SEQ ID NO: 1, a CDR2 region represented by an amino acid sequences shown in SEQ ID NO: 2 and a CDR3 region represented by an amino acid sequences shown in SEQ ID NO: 3 and a light chain variable domain comprising a CDR1 region represented by an amino acid sequences shown in SEQ ID NO: 4, a CDR2 region represented by an amino acid sequence ser-ala-ser and a CDR3 region represented by an amino acid sequences shown in SEQ ID NO: 5.

Item 29 The anti-GDF-15 antibody for use according to item 28, wherein the anti-GDF-15 antibody has a heavy chain variable domain comprising the amino acid sequence represented by SEQ ID NO: 6 or an amino acid sequence having at least 90% identity, preferably at least 95% identity and more preferably at least 98% identity to the amino acid sequence shown in SEQ ID NO: 6 and a light chain variable domain comprising the amino acid sequence represented by SEQ ID NO: 7 or an amino acid sequence having at least 90% identity, preferably at least 95% identity and more preferably at least 98% identity to the amino acid sequence shown in SEQ ID NO: 7.

Item 30 The anti-GDF-15 antibody for use according to item 28 or 29, wherein the anti-GDF-15 antibody has a heavy chain comprising the amino acid sequence represented by SEQ ID NO: 8 or an amino acid sequence having at least 90% identity, preferably at least 95% identity and more preferably at least 98% identity to the amino acid sequence shown in SEQ ID NO: 8 and a light chain comprising the amino acid sequence represented by SEQ ID NO: 9 or an amino acid sequence having at least 90% identity, preferably at least 95% identity and more preferably at least 98% identity to the amino acid sequence shown in SEQ ID NO: 9.

Item 31 The anti-GDF-15 antibody for use according to any one of items 28 to 30, wherein the anti-GDF-15 antibody has a heavy chain comprising the amino acid sequence represented by SEQ ID NO: 8 and a light chain comprising the amino acid sequence represented by SEQ ID NO: 9.

Item 32 The anti-GDF-15 antibody for use according to any one of items 1 to 31, wherein said anti-GDF-15 antibody is obtainable by expression in CHO cells.

Item 33 The anti-GDF-15 antibody for use according to any one of items 1 to 32, wherein said anti-GDF-15 antibody is visugromab.

Item 34 The anti-GDF-15 antibody for use according to any one of items 1 to 33, wherein said neoadjuvant therapy further comprises at least one therapeutic modality selected form the group consisting of cell therapy, chemotherapy, targeted therapy, immunotherapy, therapeutic vaccination or radiotherapy.

Item 35 Combination product comprising an anti-GDF-15 antibody according to any one of items 1-34 and at least one active substance as used in cell therapy, chemotherapy, targeted therapy, immunotherapy, therapeutic vaccination or radiotherapy for use in a neoadjuvant therapy of cancer in a human patient.

Item 36 A pharmaceutical composition comprising a combination of an anti-GDF-15 antibody according to any one of items 1-34 and at least one active substance as used in cell therapy, chemotherapy, targeted therapy, immunotherapy, therapeutic vaccination or radiotherapy for use in a neoadjuvant therapy of cancer in a human patient.

Item 37 The combination product according to item 35 or the pharmaceutical composition according to item 36, wherein the active substance is a checkpoint inhibitor; wherein the checkpoint inhibitor is preferably an anti-PD-1 antibody or an anti-PD-L1 antibody.

Item 38 The combination product according to item 35 or the pharmaceutical composition according to item 36, wherein the active substance is a bi-specific T-cell engager.

Item 39 The combination product according to item 35 or the pharmaceutical composition according to item 36, wherein the active substance is cell-based as used in cell therapy.

Item 40 The combination product or the pharmaceutical composition according to any one of items 35 to 39, wherein the cancer is a solid cancer, and wherein neoadjuvant therapy is for treating or inhibiting the growth of a tumor of the solid cancer.

Item 41 The combination product or the pharmaceutical composition according to item 40, wherein the patient is scheduled for surgery of a primary tumor of the solid cancer.

Item 42 The combination product or the pharmaceutical composition according to any one of items 35 to 41, wherein the surgery is a resection surgery.

Item 43 The combination product or the pharmaceutical composition according to any one of items 35 to 42, wherein said resection surgery is a radical cystectomy comprising the removal of the bladder, adjacent organs, and regional lymph nodes.

Item 44 The combination product or the pharmaceutical composition according to any one of items 35 to 43, wherein the duration of said neoadjuvant therapy prior to resection surgery is up to 3, 6 or 12 weeks.

Item 45 The combination product or the pharmaceutical composition according to any one of items 35 to 44, wherein the therapy is intended to downstage locally advanced solid cancer to reduce the extent of surgery or to perform an organ-conserving surgery.

Item 46 The combination product or the pharmaceutical composition according to item 45, wherein the duration of said neoadjuvant therapy is up to 3, 4, 6, 12 or 24 weeks.

Item 47 The combination product or the pharmaceutical composition according to any one of items 35 to 46, wherein the cancer is selected from the group consisting of colorectal cancer, gastric cancer, bladder cancer, melanoma, non-small cell lung cancer, head and neck squamous cell cancer, hepatocellular cancer, prostate adenocarcinoma, pancreatic cancer, uterine cancer, cervical cancer, thyroid cancer, cutaneous squamous cell cancer, mesothelioma, cancer of unknown primary (CUP) and breast carcinoma.

Item 48 The combination product or the pharmaceutical composition according to item 47, wherein the cancer is bladder cancer.

Item 49 The combination product or the pharmaceutical composition according to item 48, wherein the bladder cancer is muscle invasive bladder cancer.

Item 50 The combination product or the pharmaceutical composition according to any one of items 35 to 49, wherein the patient cannot receive or refuses to receive chemotherapy.

Item 51 The combination product or the pharmaceutical composition according to item 50, wherein the chemotherapy is a chemotherapy for use in neoadjuvant therapy, such as is a cisplatin-based chemotherapy.

Item 52 The combination product or the pharmaceutical composition according to any one of items 35 to 51, wherein a tissue sample of the tumor has a PD-L1 combined positive score (CPS) of ≥10%.

Item 53 The combination product or the pharmaceutical composition according to any one of items 35 to 51, wherein a tissue sample of the tumor has a PD-L1 CPS of <10%.

Item 54 The combination product or the pharmaceutical composition according to any one of items 35 to 53, wherein the cancer is classified as a T2 stage tumor.

Item 55 The combination product or the pharmaceutical composition according to any one of items 35 to 53, wherein the cancer is classified as T3/T4 stage tumor.

Item 56 The combination product or the pharmaceutical composition according to any one of items 35 to 55, wherein the cancer tissue encompasses non-basal/squamous histology.

Item 57 The combination product or the pharmaceutical composition according to any one of items 35 to 55, wherein the cancer tissue encompasses basal/squamous histology.

Item 58 The combination product or the pharmaceutical composition according to any one of items 35 to 57, wherein the anti-GDF-15 antibody is to be administered to the human patient, preferably

at a dose of between 3 and 20 mg/kg, preferably at a dose of 10 mg/kg, and at a dosage regimen of at least one administration cycle, wherein the cycle is a period of two weeks and wherein said dose is to be administered at least once in each of the at least one cycle,

at a dose of between 3 and 20 mg/kg, preferably at a dose of 10 mg/kg, and at a dosage regimen of at least one administration cycle, wherein the cycle is a period of three weeks and wherein said dose is to be administered at least once in each of the at least one cycle, or

at a dose of between 3 and 20 mg/kg, preferably at a dose of 20 mg/kg, and at a dosage regimen of at least one administration cycle, wherein the cycle is a period of four weeks and wherein said dose is to be administered at least once in each of the at least one cycle.

Item 59 The combination product or the pharmaceutical composition according to item 57, wherein the anti-GDF-15 antibody is to be administered at a dose of 20 mg/kg, and at a dosage regimen of at least one administration cycle, wherein the cycle is a period of four weeks and wherein said dose is to be administered at least once in each of the at least one cycle.

Item 60 The combination product or the pharmaceutical composition according to any one of item 57 or 58, wherein the cycle is a period of four weeks, wherein said dose is to be administered at least once in each of the at least one cycle and wherein the dose is to be administered for at least one, two, three or four cycle(s).

Item 61 The combination product or the pharmaceutical composition according to any one of item 57 to 60, wherein the anti-GDF-15 antibody is to be administered to the human patient at a dose of 20 mg/kg, and at a dosage regimen of three administration cycles, wherein the cycle is a period of four weeks and wherein said dose is to be administered at least once in each cycle.

Item 62 The combination product or the pharmaceutical composition according to any one of item 35 to 61, wherein said checkpoint inhibitor is to be administered in the same dosage regimen as the anti-GDF-15 antibody.

Item 63 The combination product or the pharmaceutical composition according to item 62, wherein said checkpoint inhibitor is to be administered prior to the administration of anti-GDF-15 antibody, preferably within 120 min prior to the administration of anti-GDF-15 antibody, more preferably within 30 minutes prior to the administration of anti-GDF-15 antibody.

Item 64 The combination product or the pharmaceutical composition according to item 62 or 63, wherein said dose of the anti-GDF-15 antibody is to be administered intravenously.

Item 65 The combination product or the pharmaceutical composition according to any one of items 35 to 64, wherein the anti-GDF-15 antibody comprises a heavy chain variable domain comprising a CDRI region represented by an amino acid sequences shown in SEQ ID NO: 1, a CDR2 region represented by an amino acid sequences shown in SEQ ID NO: 2 and a CDR3 region represented by an amino acid sequences shown in SEQ ID NO: 3 and a light chain variable domain comprising a CDRI region represented by an amino acid sequences shown in SEQ ID NO: 4, a CDR2 region represented by an amino acid sequence ser-ala-ser and a CDR3 region represented by an amino acid sequences shown in SEQ ID NO: 5.

Item 66 The combination product or the pharmaceutical composition according to item 65, wherein the anti-GDF-15 antibody has a heavy chain variable domain comprising the amino acid sequence represented by SEQ ID NO: 6 or an amino acid sequence having at least 90% identity, preferably at least 95% identity and more preferably at least 98% identity to the amino acid sequence shown in SEQ ID NO: 6 and a light chain variable domain comprising the amino acid sequence represented by SEQ ID NO: 7 or an amino acid sequence having at least 90% identity, preferably at least 95% identity and more preferably at least 98% identity to the amino acid sequence shown in SEQ ID NO: 7.

Item 67 The combination product or the pharmaceutical composition according to items 65 or 66, wherein the anti-GDF-15 antibody has a heavy chain comprising the amino acid sequence represented by SEQ ID NO: 8 or an amino acid sequence having at least 90% identity, preferably at least 95% identity and more preferably at least 98% identity to the amino acid sequence shown in SEQ ID NO: 8 and a light chain comprising the amino acid sequence represented by SEQ ID NO: 9 or an amino acid sequence having at least 90% identity, preferably at least 95% identity and more preferably at least 98% identity to the amino acid sequence shown in SEQ ID NO: 9.

Item 68 The combination product or the pharmaceutical composition according to any one of items 65 to 67, wherein the anti-GDF-15 antibody has a heavy chain comprising the amino acid sequence represented by SEQ ID NO: 8 and a light chain comprising the amino acid sequence represented by SEQ ID NO: 9.

Item 69 The combination product or the pharmaceutical composition according to any one of items 35 to 68, wherein said anti-GDF-15 antibody is obtainable by expression in CHO cells.

Item 70 The combination product or the pharmaceutical composition according to any one of items 35 to 69, wherein said anti-GDF-15 antibody is visugromab.

### BRIEF DESCRIPTION OF DRAWINGS

FIG. 1 is a flow chart describing the study design in Example 1.

FIG. 2A-FIG. 2B: Urothelial cancer Ph2a antitumoral activity: Deep and lasting responses

Antitumoral activity of a combination treatment of GDF-15 blocking antibody visugromab and nivolumab in advanced-stage, anti-PD1/PD-L1 relapsed/refractory patients with urothelial cancer. Patient response and treatment course is shown for 27 patients within the urothelial cancer ph2a cohort. The objective response rate (ORR) in urothelial cancer is 14.7% (4/27). The durability of response (DoR) is 10+ months and still maturing.

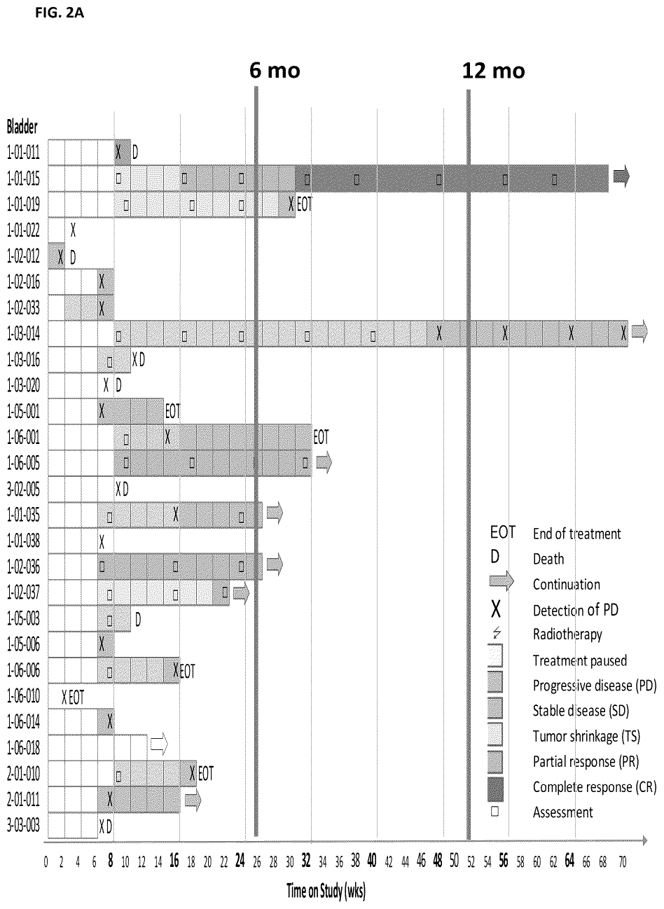

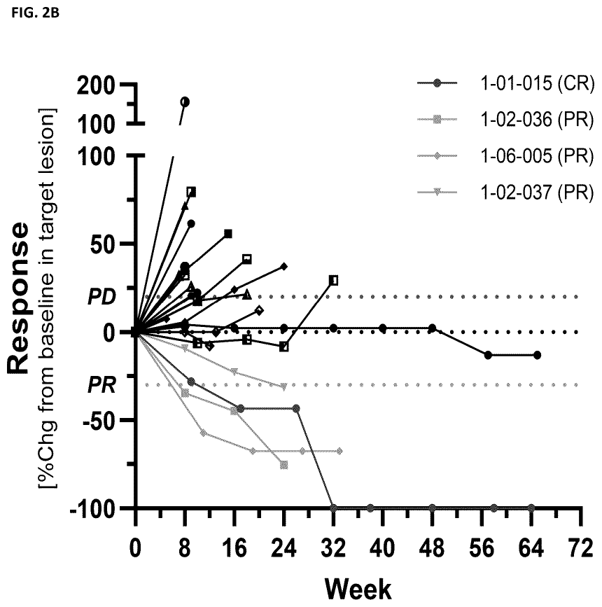

Response and treatment course of patients is shown in Swimmer's (FIG. 2A) and Spaghetti plot (FIG. 2B).

### DETAILED DESCRIPTION OF INVENTION

In a non-limiting embodiment, the neoadjuvant therapy of the present invention using an anti-GDF-15 antibody is not particularly limited and may further comprises at least one therapeutic modality selected form the group consisting of cell therapy, chemotherapy, targeted therapy, immunotherapy, therapeutic vaccination or radiotherapy.

In this respect, the neoadjuvant therapy using an anti-GDF-15 antibody further comprises the use of at least one active substance selected from the group consisting of a checkpoint inhibitor, an oncolytic virus, a bispecific engager, or an antibody-drug conjugate, such as enhertu.

It is understood that in accordance with the present invention, references to “at least one checkpoint inhibitor” mean that the indicated checkpoint inhibitor is required, and that additional checkpoint inhibitors can be included or not, i.e., they are optionally included. For example, it is understood that in cases where the present invention refers to at least one checkpoint inhibitor wherein the checkpoint inhibitor is selected from the group consisting of anti-PD-1 antibody or a PD-1-binding fragment thereof and anti-PD-LI antibody or a PD-L1-binding fragment thereof, this means that a checkpoint inhibitor selected from the group consisting of anti-PD-1 antibody or a PD-1-binding fragment thereof and anti-PD-L1 antibody or a PD-L1-binding fragment thereof is required, and that additional checkpoint inhibitors (e.g., anti-CD40 antibody or a CD40-binding fragment thereof, anti-LAG-3 antibody or a LAG-3-binding fragment thereof, anti-TIM-3 antibody or a TIM-3-binding fragment thereof, anti-TIGIT antibody or a TIGIT-binding fragment thereof and anti-CTLA4 antibody or a CTLA4-binding fragment thereof) can be included or not, i.e., they are optionally included.

In a non-limiting embodiment of the invention, the checkpoint inhibitor may be any one selected from the group consisting of an anti-PD-1 antibody, an anti-PD-L1 antibody, an anti-CD40 antibody, an anti-LAG-3 antibody, an anti-TIM-3 antibody, an anti-TIGIT antibody and an anti-CTLA4 antibody, preferably an anti-PD-1 antibody or an anti-PD-L1 antibody.

In a preferred embodiment, the anti-PD-1 antibody is selected from the group consisting of nivolumab, pembrolizumab, cemiplimab, dostarlimab, vopratelimab, spartalizumab, camrelizumab, sintilimab, tislelizumab, toripalimab, INCMGA00012 (MGA012), AMP-514 (MEDI0680) and AMP-224. In a preferred embodiment, the anti-PD-LI antibody is selected from the group consisting of atezolizumab, avelumab, durvalumab, BMS-936559, KN035, cosibelimab (CK-301), AUNP12, CA-170, BMS-986189 and MEDI4736. In a preferred embodiment, the anti-CTLA-4 antibody is ipilimumab or tremelimumab.

The administration form of the at least one checkpoint inhibitor is not particularly limited and may include for example intravenous and/or subcutaneous administration.

### DETAILED DESCRIPTION OF THE INVENTION

Unless specifically defined herein, all technical and scientific terms used herein have the same meaning as commonly understood by a skilled artisan in the fields of the invention.

All methods and materials similar or equivalent to those described herein can be used in the practice or testing of the present invention, with suitable methods and materials being described herein.

All literature references referred to herein are incorporated by reference in their entirety for all purposes. Patent applications are referred to herein by using their application and/or publication number. Non-patent literature referred to herein may be cited either as a full reference, or in abbreviated form (e.g., Danaher et al., 2018), followed by the full reference in the “References” section.

As referred to herein, each occurrence of terms such as “comprising” or “comprises” may optionally be substituted with “consisting of” or “consists of”.

As referred to herein, the term “neoadjuvant therapy” is a therapy performed prior to the surgical resection of a tumor, preferably a primary tumor. Accordingly, neoadjuvant therapy encompasses therapies intended to downstage locally advanced solid cancer to be able to reduce the extent of surgery or to perform an organ-conserving surgery. In this respect, the neoadjuvant therapy may allow to make the tumor resectable or to reduce the extent of surgery e.g., due to shrinkage of the tumor size. For example, a neoadjuvant therapy may be performed as a tumor treatment to enable a surgical resection of said tumor.

As referred to herein, it is known that some patients cannot receive or refuse to receive chemotherapy, and criteria for identifying these patients are known in the art. Preferably, a patient which “cannot receive or refuses to receive chemotherapy” refers to a patient having cisplatin ineligibility as defined per Galsky criteria shown in Table 1 below:

TABLE 1
Any one of the following:

ECOG PS ≥2, or KPS 60%-70%
Creatinine clearance <60 mL/min
CTCAE v4 grade 2 or greater peripheral neuropathy
Grade 2 or greater audiometric hearing loss
NYHA class III or greater heart failure
ECOG PS = Eastern Cooperative Oncology Group Performance Scale
KPS = Karnofsky Performance Score

As referred to herein, the term “Combined Positive Score (CPS)” refers to a score reflecting the PD-L1 expression on tumor cells and tumor-infiltrating immune cells and may be determined by a validated IHC test. For example, a combined positive score (CPS) for PD-L1 is the summed-up percentage of cancer cells and cancer-infiltrating cells (e.g., cancer-infiltrating immune cells) showing staining for the presence of PD-L1 on the cell surface. This summed-up percentage of cancer cells and cancer-infiltrating cells can be determined by counting, within the cancer sample, the sum of the numbers of all cancer cells and cancer-infiltrating cells showing staining for the presence of PD-L1 on the cell surface, and by dividing said number by the sum of the total numbers of cancer cells and cancer-infiltrating cells in the sample, and multiplying the resulting quotient by 100.

As referred to herein, the tumor classification score relates to the globally recognised standard (TNM) for classifying the extent of spread of cancer. For example, TNM classification describes the primary tumor (T), lymph node involvement (N) and evidence of metastases (M0 or M1). The T values describe the size and level of infiltration of the primary tumor.

A cancer patient to be treated according to the present invention may be selected based at least one characteristic selected from the group consisting of CPS, tumor classification score and histological cancer type.

In an aspect, the cancer tissue of a patient to be treated according to the present invention may have a PD-L1 combined positive score (CPS) of ≥10%. Said patient may further have a cancer classified as a T2 or as a T3/T4 stage tumor.

In an aspect, the cancer tissue of a patient to be treated according to the present invention may have a PD-L1 combined positive score (CPS) of <10%. Said patient may further have a cancer classified as a T2 or as a T3/T4 stage tumor.

In an aspect, the patient to be treated according to the present invention may be selected based on the histological classification of the cancer type. The skilled person in the art is aware of histological techniques for classifying cancer types based on the cellular context of the cancer. For example, carcinoma originating from the skin or in tissues that line or cover internal organs may be further classified into different cancer subtypes depending on the cell layer within the epithelial cell which is affected such as squamous cell, adenomatous cells, transitional cells and/or basal cells.

In an aspect, the cancer to be treated according to the present invention may be classified as having a basal/squamous histology.

In a further aspect, the cancer to be treated according to the present invention may be classified as having a non-basal/squamous histology.

As referred to herein, the terms “CTL-002” and visugromab are used synonymously. They refer to an antibody having the heavy chain amino acid sequence of SEQ ID NO: 8 and the light chain amino acid sequence of SEQ ID NO: 9. It is understood that any reference to the amino acid sequences of antibodies of the invention is meant to encompass posttranslational modifications of these sequences occurring in mammalian cells such as CHO cells, including, but not limited to, N-glycosylation, O-glycosylation, deamidation, Asp isomerization/fragmentation, pyro-glutamate formation, removal of C-terminal lysine, and Met/Trp oxidation.

It is understood that the antibodies according to the invention can be administered in the form of pharmaceutical compositions. Such pharmaceutical compositions are prepared in a way that they can be stored and administered appropriately, e.g. by using pharmaceutically acceptable components such as carriers, excipients or stabilizers.

Such pharmaceutically acceptable components are not toxic in the amounts used when administering the pharmaceutical composition to a human patient. The pharmaceutical acceptable components added to the pharmaceutical compositions may depend on the route of administration.

In general, the pharmaceutically acceptable components used in connection with the present invention are used in accordance with knowledge available in the art, e.g. from Remington's Pharmaceutical Sciences, Ed. AR Gennaro, 20th edition, 2000, Williams & Wilkins, PA, USA.

The invention is further illustrated by the following non-limiting examples.

### EXAMPLES

### Example 1: Neoadjuvant Immunotherapy

### Overall Study Design

This is a multi-center, stratified and single-blinded Phase 2 study of neoadjuvant immunotherapy in combination with the anti-GDF-15 antibody visugromab (CTL-002) for the treatment of subjects with MIBC set to undergo RC who cannot receive or refuses to receive cisplatin-based chemotherapy.

This exploratory study will assess and explore the clinical efficacy and safety of various immuno-oncology agents as neoadjuvant treatment of cisplatin-ineligible or cisplatin-refusing individuals with MIBC and potential for clinical benefit, as shown for e.g., anti-PD-1 inhibitors and at doses shown to add minimal/acceptable AE risk. The translational endpoints shall inform which therapies are viable candidates for further investigation in MIBC.

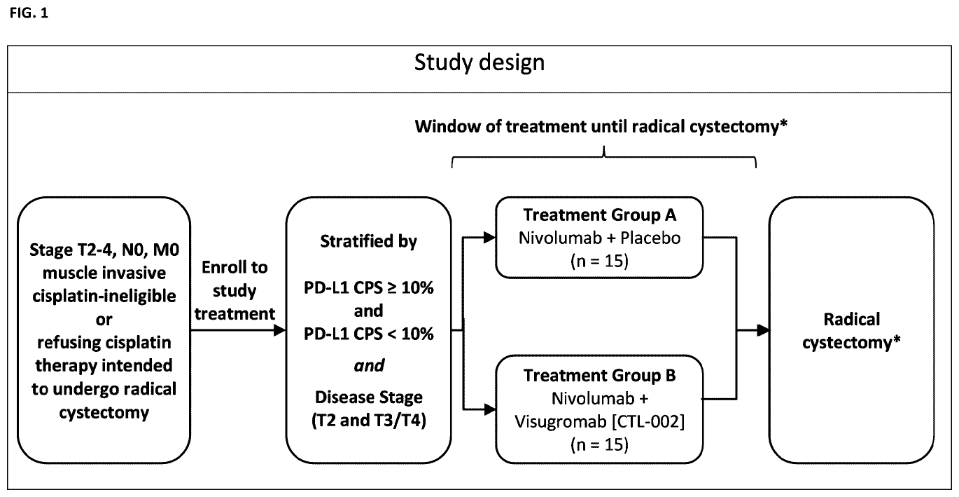

The study design is presented in FIG. 1.

The treatment of the study will be assigned and planned as follows:

Eligible subjects will be 1:1 assigned, stratified based on tumor tissue PD-L1 CPS>10% or PD-L1 CPS<10% and their tumor size (T2 or T3/T4), to one of the treatment groups

Subjects will receive study treatment as follows:
a. Treatment Group A (nivolumab+placebo)
b. Treatment Group B (nivolumab+visugromab [CTL-002])

A total of 3 cycles is planned before surgery

Curative-intent RC+pelvic lymph node dissection will be planned at the time of study inclusion to be done within 4-8 weeks from the planned last dose of study drug infusion

Efficacy assessments will be done pathologically by assessing the RC sample for pathologic response and radiologically according to RECIST v1.1. Subjects will be monitored carefully for the development of AEs and will be monitored for clinical and/or radiographic evidence of disease progression according to usual standards of clinical practice. Adverse experiences will be evaluated according to criteria outlined in the NCI-CTCAE, version 5.0.

### Immunotherapy Treatment Period

The Immunotherapy Treatment Period lasts 12 weeks (3 cycles of 4 weeks).

As indicated above, subjects will be 1:1 assigned, stratified based on tumor tissue PD-L1 CPS>10% or PD-L1 CPS<10% and their tumor size (T2 or T3/T4), to one of the following treatment groups:
Treatment Group A (nivolumab+placebo)
Treatment Group B (nivolumab+visugromab [CTL-002])

For the combination, visugromab and nivolumab will be given on the same day concomitantly, where visugromab will always be administered first, with a 30-minute observation period following the end of the visugromab (CTL-002) administration to assess safety, which will then be followed by infusion of nivolumab (Group B), administered per SmPC and local guidelines.

### Radical Cystectomy (RC)

RC entails removal of the bladder, adjacent organs, and regional lymph nodes. In males, it includes the removal of the prostate and the seminal vesicles, whereas in females, the reproductive organs (ovaries, fallopian tubes, uterus, and anterior vagina) may also be removed to achieve best oncologic outcomes. Surgery is conducted according to the hospital's standard practice (standard of care).

RC will be planned at the time of study inclusion, to be performed ideally at the end of the immunotherapy treatment period (4 to 8 weeks [28-56 days] post last study drug infusion). Tumor material will be investigated at two timepoints; first at baseline, with material of transurethral resection, and second with cystectomy 4 to 8 weeks [28-56 days] post last study drug infusion). The tumor tissue investigations are mandatory in order to assess immune cell infiltration in the tumor. If a biopsy cannot be taken for safety reasons, this should be discussed with the Medical Monitor and the Sponsor's medical representative. Subjects who refuse or are unsuited for RC at the end of the neoadjuvant period will be managed according to the European Association for Urology guidelines, and will be primarily offered a chemoradiation as a bladder-sparing option. These subjects will continue to be monitored and included in the long-term safety and efficacy analyses.

### Example 2: Antitumoral Activity of a Combination Treatment of GDF-15 Blocking Antibody visugromab and nivolumab in Bladder Cancer

A target population of advanced-stage, metastatic solid tumor patients that are relapsed/refractory to prior anti-PD1/PD-L1 treatment was selected according to the following criteria: (i) 12-wk minimum prior continuous anti-PD1/PD-L1 exposure with for indication approved anti-PD1/PD-L1 antibody and (2) either direct progression on this anti-PD1/PD-L1 treatment or (3) if initial response, seamless continuation of anti-PD1/PD-L1 treatment until relapse/progression and treated with a combination of GDF-15 blocking antibody visugromab and nivolumab.

Clinical results based on a urothelial cancer ph2a cohort support an antitumoral activity of the combination treatment of GDF-15 blocking antibody visugromab and nivolumab in advanced-stage, anti-PD1/PD-L1 relapsed/refractory patients achieving deep and lasting responses reaching up to complete response for over 1 year duration (see FIG. 2A-FIG. 2B).

In FIG. 2A (Swimmer's plot), clinical results of 27 patients with urothelial cancer are depicted over time showing an objective response rate (ORR) of 14.7% [4/27]. The DoR is 10+ months and still maturing.

For example, patient [1-01-15] showed tumor shrinkage (TS) from week 8-16, followed by partial response (PR) in weeks 16-30 and complete response (CR) as of week 32. Patient [1-01-019] showed tumor shrinkage (TS) as of week 8. Patient [1-06-005] showed partial response (PR) as of week 10. Patient [1-02-036] showed partial response (PR) as of week 6. Patient [1-02-037] showed tumor shrinkage (TS) from week 6-20 and partial response (PR) as of week 20.

In FIG. 2B (Spaghetti plot), the response, i.e. % change from baseline in target lesion, of various patients is depicted over time. Response curves of patients with complete (CR) or partial response (PR) are located on the negative range of the ordinate axis as exemplified by curves of patients [1-01-015], [1-02-035], [1-06-005], [1-02-037].

In summary, in the urothelial cancer cohort, a response rate of 14.8% and no grade 4/5 AEs (so clean safety profile) was shown. In addition, the data also support an exceptional durability of response (DoR) of 10+ months with patients still in PR/CR further elongating DoR.

According to the invention, it is expected that the antitumoral activity of the anti-GDF-15 antibody (e.g. particularly visugromab) and its capability to reinvigorate an immune response to the cancer (in particular bladder cancer) will allow effective neoadjuvant therapy. For example, administration of the anti-GDF-15 antibody will help to stimulate an immune response against the tumor prior to resection which will allow destruction of remaining tissue post-surgery and micrometastases subsequently reducing the risk of relapse after surgery. This applies particularly also in the presence of immune checkpoint inhibitors such as anti-PD-1 or anti-PD-L1 antibodies sustaining an existing antitumoral immune response. Further, according to the invention, the advantageous safety profile of the anti-GDF-15 antibody (e.g. particularly visugromab) makes the anti-GDF-15 antibody suitable for the neoadjuvant therapy setting.

Sequences:
(Heavy Chain CDR1 Region Peptide
Sequence of monoclonal anti-
human GDF-15 antibody):
SEQ ID No: 1
GFSLSTSGMG
(Heavy Chain CDR2 Region Peptide Sequence
of monoclonal anti-
human GDF-15 antibody):
SEQ ID No: 2
IYWDDDK
(Heavy Chain CDR3 Region Peptide Sequence
of monoclonal anti-
human GDF-15 antibody):
SEQ ID No: 3
ARSSYGAMDY
(Light Chain CDR1 Region Peptide Sequence
of monoclonal anti-human GDF-15 antibody):
SEQ ID No: 4
QNVGTN
Light Chain CDR2 Region Peptide Sequence of
monoclonal anti-human GDF-15 antibody:
SAS
(Light Chain CDR3 Region Peptide Sequence of
monoclonal anti-human GDF-15 antibody):
SEQ ID No: 5
QQYNNFPYT
(heavy chain variable domain of monoclonal
anti-human GDF-15 antibody):
SEQ ID No: 6
QITLKESGPTLVKPTQTLTLTCTFSGFSLSTSGMGVSWIRQPPGKGLEWL
AHIYWDDDKRYNPTLKSRLTITKDPSKNQVVLTMTNMDPVDTATYYCARS
SYGAMDYWGQGTLVTVSSASTKGP
(light chain variable domain of monoclonal
anti-human GDF-15 antibody):
SEQ ID No: 7
DIVLTQSPSFLSASVGDRVTITCKASQNVGTNVAWFQQKPGKSPKALIYS
ASYRYSGVPDRFTGSGSGTEFTLTISSLQPEDFAAYFCQQYNNFPYTFGG
GTKLEIKRT
(heavy chain of monoclonal anti-human GDF-15
antibody CTL-002 without the leader
peptide sequence):
SEQ ID No: 8
QITLKESGPTLVKPTQTLTLTCTFSGFSLSTSGMGVSWIRQPPGKGLEWL
AHIYWDDDKRYNPTLKSRLTITKDPSKNQVVLTMTNMDPVDTATYYCARS
SYGAMDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLG
(light chain of monoclonal anti-human GDF-15
antibody CTL-002 without the leader
peptide sequence):
SEQ ID No: 9
DIVLTQSPSFLSASVGDRVTITCKASQNVGTNVAWFQQKPGKSPKALIYS
ASYRYSGVPDRFTGSGSGTEFTLTISSLQPEDFAAYFCQQYNNFPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
(heavy chain variable domain of anti-human
GDF-15 antibody H1L5):
SEQ ID NO: 10
QITLKESGPTLVKPTQTLTLTCTFSGFSLSTSGMGVSWIRQPPGKGLEWL
AHIYWDDDKRYNPTLKSRLTITKDPSKNQVVLTMTNMDPVDTATYYCARS
SYGAMDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG
(light chain variable domain of anti-human
GDF-15 antibody H1L5):
SEQ ID NO: 11
DIVLTQSPSFLSASVGDRVTITCKASQNVGTNVAWFQQKPGKSPKALIYS
ASYRYSGVPDRFTGSGSGTEFTLTISSLQPEDFAAYFCQQYNNFPYTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### REFERENCES

von der Maase, Hans, et al. “Long-term survival results of a randomized trial comparing gemcitabine plus cisplatin, with methotrexate, vinblastine, doxorubicin, plus cisplatin in patients with bladder cancer.” Journal of clinical oncology 23.21 (2005): 4602-4608.

Grossman, H. Barton, et al. “Neoadjuvant chemotherapy plus cystectomy compared with cystectomy alone for locally advanced bladder cancer.” New England Journal of Medicine 349.9 (2003): 859-866.

### INDUSTRIAL APPLICABILITY

The anti-GDF-15 antibody used in a neoadjuvant treatment of cancer in human patients can be industrially manufactured and sold as products for the itemed methods and uses, in accordance with known standards for the manufacture of pharmaceutical products. Accordingly, the present invention is industrially applicable.

1.-70. canceled

71. A method of neoadjuvant therapy for treating cancer in a human patient, the method comprising administering to the human patient a combination of an anti-GDF-15 antibody and at least one additional active substance, wherein the additional active substance is a substance used in cell therapy, chemotherapy, targeted therapy, immunotherapy, therapeutic vaccination, or radiotherapy.

72. The method of claim 71, wherein the anti-GDF-15 antibody and the additional active substance are administered in a combination product or composition.

73. The method of claim 71, wherein the additional active substance is:
(a) a checkpoint inhibitor;
(b) a bi-specific T-cell engager; or
(c) a cell-based therapy.

74. The method of claim 73, wherein the checkpoint inhibitor is an anti-PD-1 antibody or an anti-PD-L1 antibody.

75. The method of claim 71, wherein the cancer is a solid cancer.

76. The method of claim 75, wherein the neoadjuvant therapy treats or inhibits the growth of the solid cancer.

77. The method of claim 75, wherein the patient is scheduled for a resection surgery of a primary tumor of the solid cancer, optionally wherein said resection surgery is a radical cystectomy comprising the removal of the bladder, adjacent organs, and regional lymph nodes.

78. The method of claim 77, wherein:
(a) the neoadjuvant therapy is administered up to 12 weeks prior to resection surgery; and/or
(b) the neoadjuvant therapy reduces the extent of the resection surgery.

79. The method of claim 75, wherein the solid cancer is selected from the group consisting of colorectal cancer, gastric cancer, bladder cancer, melanoma, non-small cell lung cancer, head and neck squamous cell cancer, hepatocellular cancer, prostate adenocarcinoma, pancreatic cancer, uterine cancer, cervical cancer, thyroid cancer, cutaneous squamous cell cancer, mesothelioma, cancer of unknown primary (CUP), and breast carcinoma.

80. The method of claim 79, wherein the solid cancer is muscle invasive bladder cancer.

81. The method of claim 71, wherein the human patient cannot receive or refuses to receive chemotherapy, optionally wherein the chemotherapy is cisplatin-based chemotherapy.

82. The method of claim 77, wherein:
(a) a tissue sample of the primary tumor has a PD-L1 combined positive score (CPS) of >10%;
(b) the cancer is classified as a T2 stage tumor; and/or
(c) cancer tissue from the patient encompasses non-basal/squamous histology.

83. The method of claim 77, wherein:
(a) a tissue sample of the primary tumor has a PD-L1 combined positive score (CPS) of <10%;
(b) the cancer is classified as a T3/T4 stage tumor; and/or
(c) cancer tissue from the patient encompasses basal/squamous histology.

84. The method of claim 71, wherein the anti-GDF-15 antibody is administered to the human patient at a dose of between 3 and 20 mg/kg and at a dosage regimen of at least one administration cycle, wherein the cycle is a period of two, three, or four weeks, and wherein the dose is administered at least once in each of the at least one administration cycle.

85. The method of claim 84, wherein:
(a) the administration cycle is a period of two or three weeks and the dose is 10 mg/kg; or
(b) the administration cycle is a period of four weeks and the dose is 20 mg/kg.

86. The method of claim 84, wherein the dosage regimen is three administration cycles.

87. The method of claim 74, wherein:
(a) the checkpoint inhibitor is administered at the same dosage regimen as the anti-GDF-15 antibody, optionally within 120 min prior to administration of the anti-GDF-15 antibody; and/or
(b) the anti-GDF-15 antibody is administered intravenously.

88. The method of claim 71, wherein the anti-GDF-15 antibody comprises:
(a) a heavy chain variable domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3, and a light chain variable domain comprising a CDR1 comprising the amino acid sequence of SEQ ID NO: 4, a CDR2 comprising the amino acid sequence of ser-ala-ser, and a CDR3 region comprising the amino acid sequence of SEQ ID NO: 5;
(b) a heavy chain variable domain comprising the amino acid sequence of SEQ ID NO: 6 or an amino acid sequence having at least 90% identity to SEQ ID NO: 6, and a light chain variable domain comprising the amino acid sequence represented by SEQ ID NO: 7 or an amino acid sequence having at least 90% identity to SEQ ID NO: 7;
(c) a heavy chain comprising the amino acid sequence of SEQ ID NO: 8 or an amino acid sequence having at least 90% identity to SEQ ID NO: 8 and a light chain comprising the amino acid sequence represented by SEQ ID NO: 9 or an amino acid sequence having at least 90% identity to SEQ ID NO: 9; or
(d) a heavy chain consisting of the amino acid sequence of SEQ ID NO: 8 and a light chain consisting of the amino acid sequence of SEQ ID NO: 9.

89. The method of claim 71, wherein:
(a) the anti-GDF-15 antibody was produced by expression in CHO cells; and/or
(b) the anti-GDF-15 antibody is visugromab.

90. The method of claim 71, wherein the neoadjuvant therapy further comprises at least one additional therapeutic modality selected from the group consisting of cell therapy, chemotherapy, targeted therapy, immunotherapy, therapeutic vaccination, and radiotherapy.


SEQUENCE LISTING (11 sequences)

SEQ ID NO: 1 - protein, 10 aa, synthetic construct
  REGION 1..10: note = Heavy Chain CDR1 Region Peptide Sequence of monoclonal anti-human GDF-15 antibody
GFSLSTSGMG

SEQ ID NO: 2 - protein, 7 aa, synthetic construct
  REGION 1..7: note = Heavy Chain CDR2 Region Peptide Sequence of monoclonal anti-human GDF-15 antibody
IYWDDDK

SEQ ID NO: 3 - protein, 10 aa, synthetic construct
  REGION 1..10: note = Heavy Chain CDR3 Region Peptide Sequence of monoclonal anti-human GDF-15 antibody
ARSSYGAMDY

SEQ ID NO: 4 - protein, 6 aa, synthetic construct
  REGION 1..6: note = Light Chain CDR1 Region Peptide Sequence of monoclonal anti-human GDF- 15 antibody
QNVGTN

SEQ ID NO: 5 - protein, 9 aa, synthetic construct
  REGION 1..9: note = Light Chain CDR3 Region Peptide Sequence of monoclonal anti-human GDF- 15 antibody
QQYNNFPYT

SEQ ID NO: 6 - protein, 124 aa, synthetic construct
  REGION 1..124: note = heavy chain variable domain of monoclonal anti-human GDF-15 antibody
QITLKESGPTLVKPTQTLTLTCTFSGFSLSTSGMGVSWIRQPPGKGLEWLAHIYWDDDKRYNPTLKSRLTITKDPSKNQVVLTMTNMDPVDTATYYCARSSYGAMDYWGQGTLVTVSSASTKGP

SEQ ID NO: 7 - protein, 109 aa, synthetic construct
  REGION 1..109: note = light chain variable domain of monoclonal anti-human GDF-15 antibody
DIVLTQSPSFLSASVGDRVTITCKASQNVGTNVAWFQQKPGKSPKALIYSASYRYSGVPDRFTGSGSGTEFTLTISSLQPEDFAAYFCQQYNNFPYTFGGGTKLEIKRT

SEQ ID NO: 8 - protein, 444 aa, synthetic construct
  REGION 1..444: note = heavy chain of monoclonal anti-human GDF-15 antibody CTL-002 without the leader peptide sequence
QITLKESGPTLVKPTQTLTLTCTFSGFSLSTSGMGVSWIRQPPGKGLEWLAHIYWDDDKRYNPTLKSRLTITKDPSKNQVVLTMTNMDPVDTATYYCARSSYGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - protein, 214 aa, synthetic construct
  REGION 1..214: note = light chain of monoclonal anti-human GDF-15 antibody CTL-002 without the leader peptide sequence
DIVLTQSPSFLSASVGDRVTITCKASQNVGTNVAWFQQKPGKSPKALIYSASYRYSGVPDRFTGSGSGTEFTLTISSLQPEDFAAYFCQQYNNFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 447 aa, synthetic construct
  REGION 1..447: note = heavy chain variable domain of anti-human GDF-15 antibody H1L5
QITLKESGPTLVKPTQTLTLTCTFSGFSLSTSGMGVSWIRQPPGKGLEWLAHIYWDDDKRYNPTLKSRLTITKDPSKNQVVLTMTNMDPVDTATYYCARSSYGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 214 aa, synthetic construct; identical to SEQ ID NO: 9
  REGION 1..214: note = light chain variable domain of anti-human GDF-15 antibody H1L5